(19) United States
(12) Patent Application Publication
(10) Pub. No.: US 2026/0057970 A1
(43) Pub. Date: Feb. 26, 2026

(54) PREDICTIVE METHOD BASED UPON MACHINE LEARNING FOR THE DEVELOPMENT OF COMPOSITES FOR TIRE TREAD COMPOUNDS

(71) Applicant: Bridgestone Europe NV/SA, Zaventem (BE)

(72) Inventors: Paolo Paciotta, Roma (IT); Andrea Guidi, Roma (IT); Roberto Lombardi, Roma (IT)

(21) Appl. No.: 18/702,861

(22) PCT Filed: Nov. 23, 2022
(86) PCT No.: PCT/IB2022/061318

§ 371 Date: Apr. 19, 2024

(30) Foreign Application Priority Data
Nov. 23, 2021 (IT) 102021000029534

(51) Int. Cl. G16C 20/30; G16C 20/70; G16C 60/00

(52) U.S. Cl. CPC G16C 20/30; G16C 20/70; G16C 60/00

(57) ABSTRACT

The present invention refers to a predictive method based upon machine learning for the development of composites for tyre tread compounds.

The present invention refers to a predictive method for the static properties of rubber compounds that is based upon machine learning, to be implemented therefore by means of an electronic computer, for the development of composites for tire tread compounds.

### BACKGROUND

The present invention is in the tire manufacturing sector, in particular with reference to the determination of the composition of those rubber compounds used for manufacturing tire treads.

The static properties of these rubber compounds (e.g. Elastic Modulus vs Elongation, Modulus at Break and Elongation at Break), at different temperature conditions and different aging conditions, play a key role in determining tire performance, in particular that related to the marketability of the product in terms of Chunk-out, Cut&Chip, Tear and High Speed Resistance. Furthermore, the Elastic Modulus at specific deformations is a key parameter to ensure certain process steps in the plant (e.g. rubber extrusion and tire construction).

These properties are ensured by the characteristics of the recipes used for the composites, in particular in terms of the ingredients, the quantity thereof and the particular synergies that are established between two or more thereof.

Commonly, the correct formulation of the recipes used for composites must go through several validation steps in the laboratory in order to first find the right technological package and then optimize the formulation by means of progressive fine-tuning until the objective is fully achieved.

Each of these iterative experimental campaigns leads, from the product point of view, to an increase in lead-times and costs in developing the product (time to market) and, from the data point of view, to the generation of a database with intrinsic variability due to random noise within the measurements made during the various test campaigns.

Prediction of product performance, in the terms outlined above, typically requires extensive laboratory testing to achieve compound validation and requires time and resources.

In particular, an object of the present invention is that of simulating laboratory tests, in order to provide an accurate estimate of some of the significant static properties of composites for the production of rubber compounds for tires without the need to perform any physical tests.

Further characteristics of the present invention are defined in the corresponding dependent claims.

The use of a software tool that may predict the behaviour of composites, and therefore tire performance, allows for:
a significant reduction in recurring costs (raw materials, labour, etc.);
optimized execution capacity and quality of laboratory tests (making it possible to allocate manpower to other activities);
shorter time to market for new products;
increased predictive precision in relation to known methodologies.

Other clear advantages over the prior art, together with the characteristics and usages of the present invention, will become clear from the following detailed description of the preferred embodiments thereof, given purely by way of a non-limiting example.

### BRIEF DESCRIPTION OF THE FIGURES

Reference will be made to the drawings in the attached figures, wherein:

FIGS. 1A, 1B, 1C illustrate by way of example the process of the present invention;

FIG. 2 shows a block diagram of a machine learning algorithm usable according to the present invention;

FIG. 3 is an example of a scatter plot of the values of the original Modulus at Break Tb vs predicted Tb values;

FIG. 4 represents the “connections”, i.e., the possibilities of reducing the variability, between the various experimental sessions;

FIG. 5 shows a graph representing the Stress-Strain curves of rubber samples corresponding to the same recipe but under 4 different test conditions.

### DETAILED DESCRIPTION OF POSSIBLE EMBODIMENTS OF THE INVENTION

The present invention will be described below with reference to the above figures.

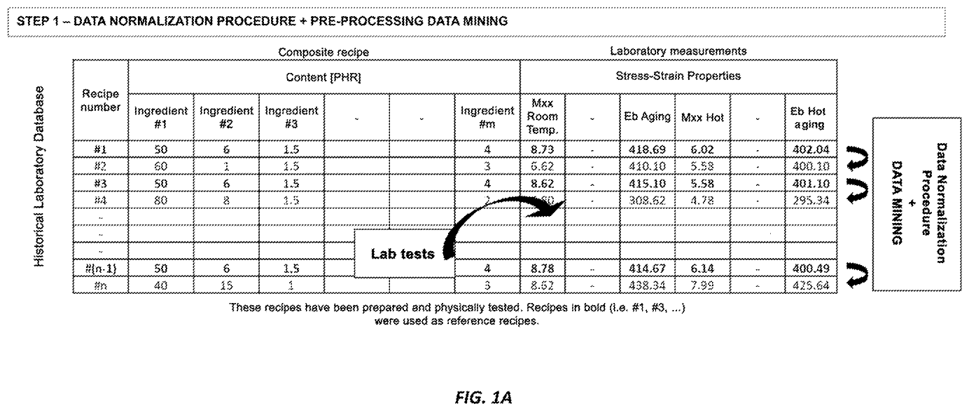

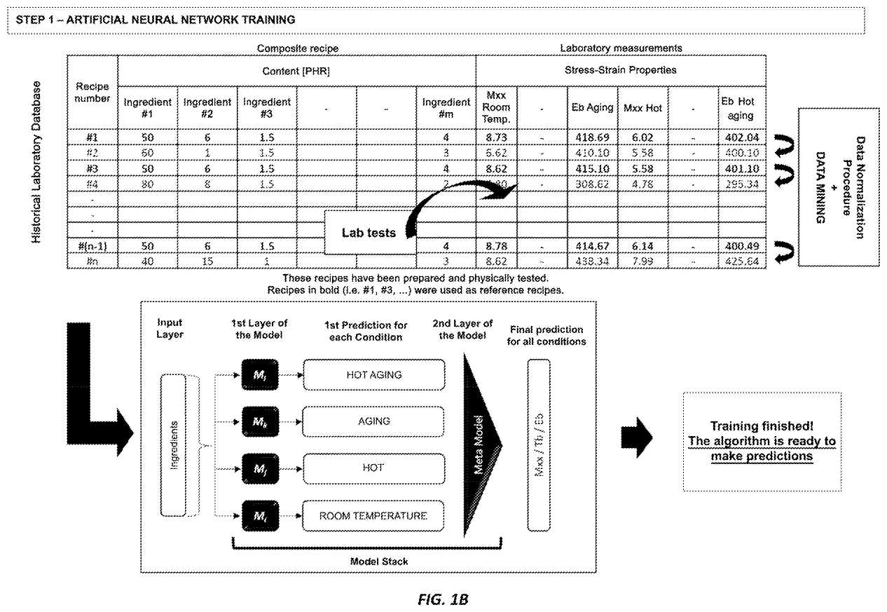

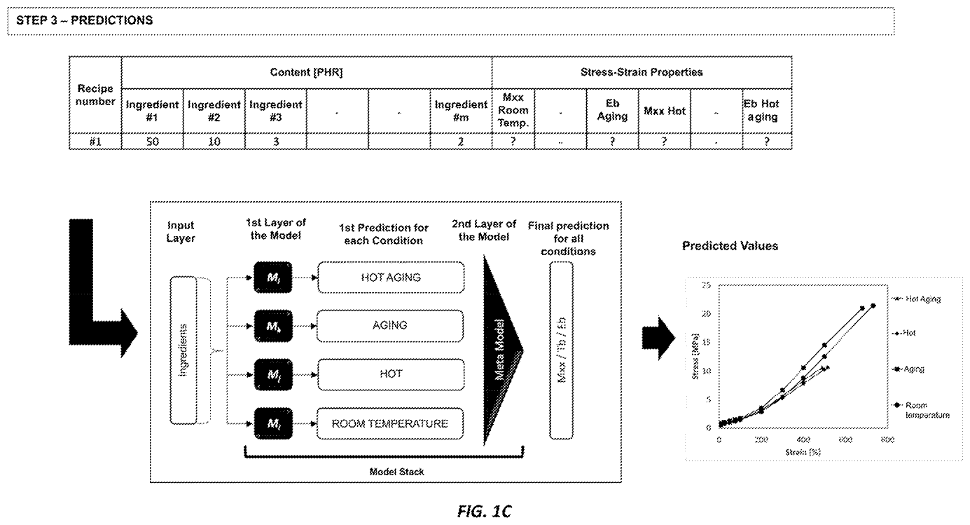

FIGS. 1A, 1B, 1C illustrate by way of example a process according to the present invention.

A methodology will therefore be described for the prediction of static properties (e.g. Elastic Modulus vs Elongation, Modulus at Break and Elongation at Break) of composites that may be used for the production of rubber compounds for tires.

In general terms, the process involves the following procedure:
generation of a raw data database, namely a dataset consisting of recipes for already existing composites and of corresponding known static properties (N experimental sessions, each containing tests on MN compounds);
a procedure for the iterative normalization of the data contained in the raw data database;
the pre-processing of the normalized data by means of Data Mining;
the training and application of an algorithm based upon automatic learning (machine learning, for example an artificial neural network), which also introduces physical constraints.

Physical constraints are understood as the physical rules that compounds must respect when subjected to a stress-strain test under different temperature (e.g. room temperature, Hot) and aging (e.g. Aging, Hot Aging) conditions.

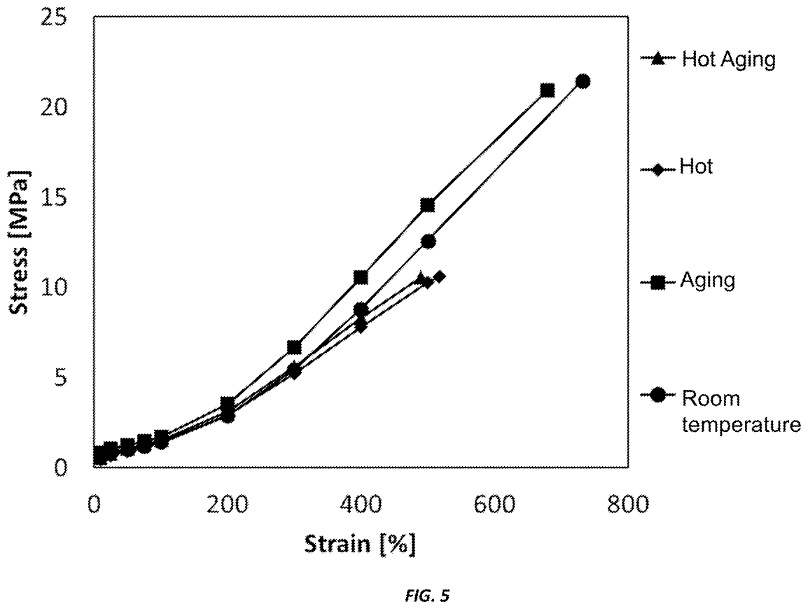

These rules, illustrated in FIG. 5, may be summarized in the following tables.

Tensile strength

| | Room | | | |
|---|---|---|---|---|
| | temperature | Aging | Hot | Hot aging |
| Room | = | > | > | > |
| temperature | | | | |
| Aging | < | = | > | > |
| Hot | < | < | = | Undefined |
| Hot aging | < | < | Undefined | = |

Elongation at break

| | Room | | | |
|---|---|---|---|---|
| | temperature | Aging | Hot | Hot aging |
| Room | = | > | > | > |
| temperature | | | | |
| Aging | < | = | > | > |
| Hot | < | < | = | Undefined |
| Hot aging | < | < | Undefined | = |

Mxx

| | Room | | | |
|---|---|---|---|---|
| | temperature | Aging | Hot | Hot aging |
| Room | = | < | > | > |
| temperature | | | | |
| Aging | > | = | > | > |
| Hot | < | < | = | Undefined |
| Hot aging | < | < | Undefined | = |

More precisely:
The aged and hot test conditions introduce an overall decrease in Tb and Eb values.
The hot test condition introduces a greater decrease in the Tb and Eb values compared to the aged test condition
The aged test condition introduces an overall increase in the M×x values
The hot test condition introduces an overall decrease of the M×x values.

In particular, the machine learning algorithm used is based on a stack of machine learning algorithms in sequence. Specifically, according to a preferred embodiment of the present invention, the stack provides for the application of two modelling layers in sequence.

The Machine Learning algorithm stack aims to perform the prediction of the static properties of rubber compounds and at the same time to apply physical constraints on the relationship between the Stress-Strain curves obtained under different test conditions.

More specifically, the first layer of the stack aims to make static property predictions specifically for each test condition. Each model (i.e. Ml, Mk, Mj, Mi) is dedicated to a specific temperature/aging condition (i.e. compound condition). Nonetheless, these predictions may lack physical coherence between them, i.e. the variabilities that are observed by carrying out the test with the same formulation, but in different physical experimental test conditions, may not be respected (see FIG. 5). The reason for this lies in the fact that the predictions were obtained from different Machine Learning models. To introduce such physical coherence, the second layer of the Machine Learning algorithm stack was developed and trained. In fact, the Machine Learning algorithms belonging to the second layer of the stack are trained by receiving as input not only the formulations (as in the previous layer), but also the static properties of the compounds estimated under different test conditions. This means that these algorithms are able to automatically learn the reciprocal relationships which may be observed between the properties as the physical test conditions vary (see FIG. 5), thus managing to imprint the necessary physical constraints automatically and implicitly.

After a step of training the model using the dataset contained in the normalized and pre-processed database as described above, it is possible to predict the static properties of the composite with greater precision than by directly applying an algorithm straight to the raw data in the database.

In fact, in this way, it is possible to drastically reduce the effect, on the predictive precision, of database noise and the intrinsic variability of the data.

In fact, by means of the procedure for the iterative normalization of the data, operating by means of data relating to multiple laboratory tests on the same species of reference (at least one of the MN compounds present in each of the N experimental sessions), the aim is to reduce the intrinsic experimental variability. Indeed, each repeated test, performed during specific experimental sessions, is used to estimate the rate of variability due to these specific experimental conditions.

Furthermore, the pre-processing procedure (Data Mining) is used to improve the accuracy of predictions by developing new capabilities, removing aberrant data and performing a principal component analysis (PCA).

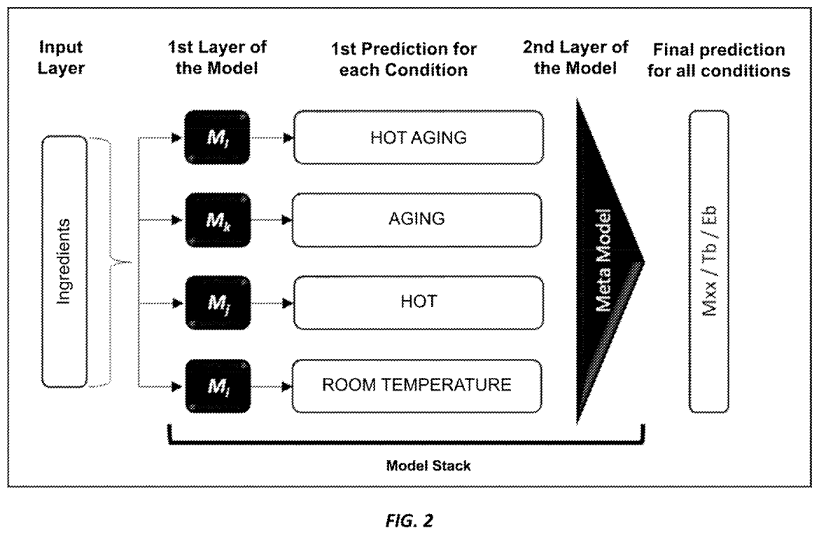

Finally, the machine learning algorithm, or rather the stack of algorithms, implemented for example through an artificial neural network (ANN), performs the prediction of some of the main static properties of the compounds under examination, for example, as already indicated, Stress-Strain curves obtained for different test conditions and different compound conditions. With reference to FIG. 2, the first layer of the model takes into account the various possible conditions, while the second layer introduces and applies physical constraints, as will be explained in greater detail below.

### Theoretical Background

Polymer matrix composite materials are unique materials, with both a characteristic elastic and viscous response when subjected to stress: the stress-strain properties of the rubber compound are usually measured by putting a sample with a characteristic dog bone shape under tension until it breaks, according to ASTM procedures.

For very low strains, the ratio between the resulting stress and the applied strain is a constant called Young's modulus in accordance with Hooke's law, valid below a certain limit, generally around 100% of the strain. As the deformation increases, the linearity breaks down, Hooke's law is no longer applicable and the rubber exhibits a non-linear increase in the value of its modulus until it breaks, releasing the stored energy: this behaviour has a great impact during different stages of the rubber manufacturing process but also during the assembly of the tire itself for various reasons.

The output of the Stress-Strain test (i.e. Stress-Strain) is a Stress Vs Strain curve from which it is possible to extract the following parameters at different test temperatures and different aging conditions of the sample:
Modulus at different strain levels (i.e. Mxx, where xx represents the strain level).
Elongation and Modulus at break (i.e. Eb and Tb, respectively).

The results are validated by comparing the values of the Stress-Strain curve, as predicted by the developed algorithm, with those known experimentally for a plurality of new experimental recipes, which were obviously not used to feed the stack of Machine Learning algorithms during the training step.

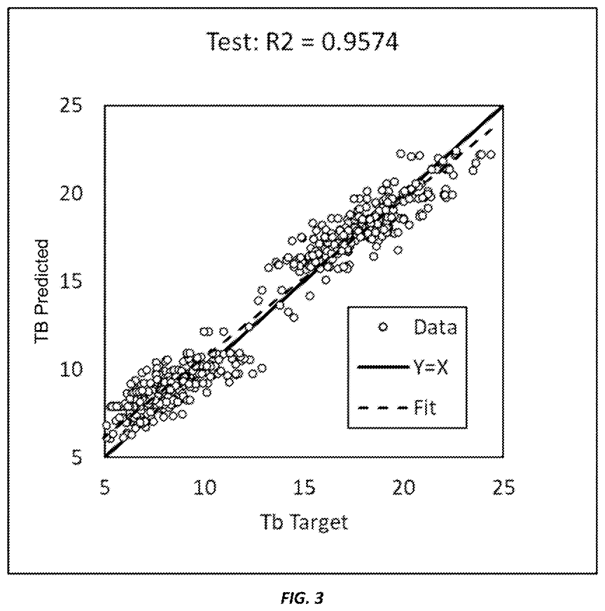

FIG. 3 shows the scatter plot of the original Tb values versus the predicted Tb values as an example of performance on the test set. As can be seen, the dispersion is characterized by a high R² value (>0.95).

It should be noted that, according to the invention, important pre-processing steps are performed before the ANN algorithm training step. More specifically, the aforementioned data normalization procedure+the pre-processing step by means of data mining.

### Iterative Data Normalization Procedure:

This normalization procedure showed the best performing improvement. In this type of application, due to the repeated experimental sessions, it is usually possible to observe high variability as regards the target properties. Indeed, some recipes are often repeated in several experimental sessions and the target properties thereof sometimes demonstrate significant differences. By investigating all of the N experimental sessions performed, it is possible to find different recipes, amongst the MN possible recipes, that may be used to reduce this variability in relation to the experimental sessions.

The normalization is carried out on each experimental session by referring to those physical properties of the recipe that are common to the various experimental sessions. If such a recipe cannot be used to normalize some of the experimental sessions, insofar as it is not included in them, a new recipe will be selected, in such a way that it is included in at least one already normalized experimental session and in those experimental sessions still to be normalized. By means of this selection it will be possible to iteratively extend and apply the normalization to new experimental sessions.

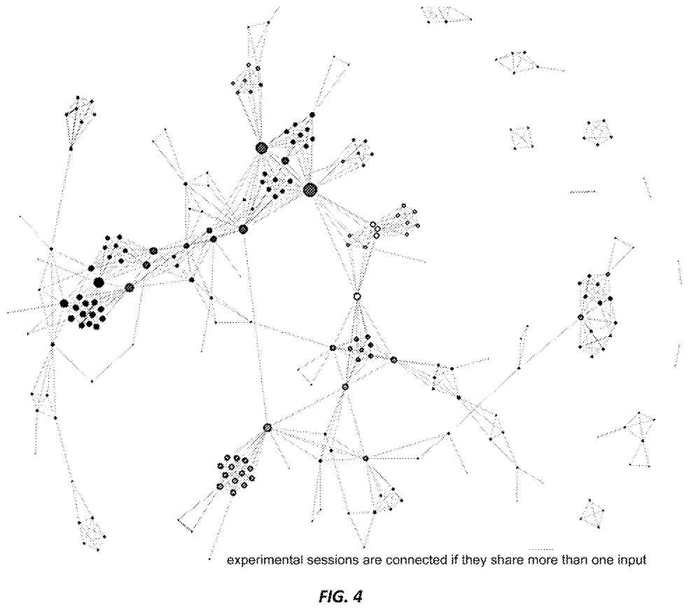

FIG. 4 shows the “connections”, namely the possibilities of reducing the variability by means of common formulations (recipes), between the various experimental sessions. The spots represent the experimental sessions, while the lines represent the “connections”, i.e., the methods of normalization of the experimental sessions by means of the reference compounds/formulations (recipes). The graph represents all possible ways to “connect” (i.e., normalize) the experimental sessions, and thus to reduce the variability thereof. As can be understood by looking at the proposed graph, each experimental session may be linked to many other sessions. Such a procedure may therefore be performed iteratively in order to reduce the variability in as many experimental sessions as possible.

From a mathematical point of view, these connections may be made in many ways and therefore different normalization procedures may be used.

According to the invention, each target property is divided by those corresponding to the recipes used as a reference in the experimental sessions.

From an operational point of view, the iterative normalization procedure is performed as follows:
1. The selection of all experimental sessions that contain the recipe F_{MR} (Most Repeated Formulation) that is most repeated in the dataset.
2. The physical properties of all the formulations included in all of the experimental sessions, selected in the previous point, are normalized by referring to the corresponding properties of the recipe F_{MR};
3. Each normalized experimental session SS_{Normalized}, is connected, according to the graph of FIG. 4, by means of a recipe F_{C} (Common Formulation), to a non-normalized experimental session SS_{NotNormalized}, therefore:
   a. The physical properties of the recipe F_{C} included in SS_{NotNormalized} are normalized by taking as reference the physical properties of F_{C} included in SS_{Normalized};
   b. The physical properties of all of the recipes included in SS_{NotNormalized} are normalized by taking as reference those physical properties of F_{C} included in SS_{NotNormalized} (which has already been pre-normalized);
4. The procedure described in point 3 is applied iteratively to all the experimental sessions according to the graph of FIG. 4.

It is important to highlight that, according to the invention and contrary to what happens in the known prior art, data normalization is not applied to the data set as a whole. The normalization procedure is applied in a specific and targeted way to each experimental session, and is developed in order to make each individual experimental session comparable to the others, thereby forming the data set as a whole. This objective is achieved by reducing the variability in relation to the experimental session. This means that what is generally discouraged in the known art, insofar as it introduces harmful non-linearities, namely the normalization of different data sets in different ways, according to the invention is used and exploited in order to achieve the desired results by means of the implementation of iterative normalizations that are determined according to the connections of the graph in FIG. 4.

The normalization procedure may be described as:

$${\overline{y}}_{i , j , k} = \frac{{y}_{i , j , k}}{{y}_{i , {ref}_{i} , k}}$$

wherein: i stands for i-th experimental session, j stands for the j-th example belonging to the specific experimental session, k stands for the k-th target property, refᵢ indicates the reference example of the i-th experimental session and y̅_{i,j,k} stands for y normalized.

The following Table 1 shows the difference between performing the data normalization procedure or not in terms of accuracy.

In doing so, accuracy is defined as the percentage of recipes that show a percentage prediction error that is lower than the target percentage error. The M100 value prediction model showed an increase in accuracy of about 30% by virtue of the application of the data normalization procedure (see the DELTA column), while the Eb and Tb value prediction models showed an increase in precision of about 26%.

TABLE 1

| | | Accuracy (% of population within the target error) | | |
|---|---|---|---|---|
| Target | % Target | Without | With | |
| property | error | normalization | normalization | DELTA |
| M100 | Err < 13% | 54.2% | 83.7% | 29.5% |
| Eb | Err < 9% | 48.2% | 74.5% | 26.3% |
| Tb | Err < 9% | 47.2% | 72.9% | 25.7% |

This table shows, by way of example, the predictive accuracy of M100, Eb and Tb in order to highlight the impact of the data normalization procedure. Normalized data processing improves the predictive performance of each individual target property. Interestingly, the normalization procedure introduces an improvement in the prediction accuracy of M100 of about 30% (from 54.2% accuracy without normalization to 83.7% accuracy with normalized data).

### Pre-Processing Using Data Mining

The accuracy of the prediction is greatly improved when a correct data mining operation (iterative normalization, aberrant data removal, PCA) is performed on the experimental dataset used to build the algorithm during the “training step”. Indeed, PCA is able to remove those ingredients that do not affect the target properties from the recipes of the training dataset and to add new fictitious ingredients, created specifically in order to emphasize the informative content of the dataset.

With the term informative contribution of a feature (and therefore, by extension, informative contribution of the dataset) reference is being made to the fact that the effect thereof upon the physical properties being predicted is well interpreted by the model, again in relation to the quantity and interaction thereof with other ingredients, in line with the performance thereof. A correct increase of 2 Mpa in relation to one property of those in question following an increase/decrease of a certain ingredient is a ratio that, if properly interpreted by the model, is a positive informative contribution.

The anomalous data removal procedure is designed to be implemented by taking into account both each individual experimental session alone and all of the various experimental sessions jointly. This dual nature of the procedure makes it possible to take advantage of every single session.

In order to add new fictitious ingredients, with the aim of facilitating the subsequent creation of predictive models, the original ingredients have been divided into certain categories, i.e., polymers, fillers, accelerators, etc. PCA was then applied to each ingredient category in order to estimate a new fictitious ingredient that could enhance the informative content of that particular ingredient category. In this context, therefore, it is possible to define as a fictitious ingredient a linear combination of the actual ingredients, as supplied to the PCA, that is such that it may emphasize the informative contribution of that specific category of ingredients. This linear combination therefore combines the informative contribution of the initial ingredients. From this it follows that the informative contribution made by the fictitious ingredient summarizes and amplifies the informative contribution of the initial ingredients. Finally, for each category of ingredients, the fictitious ingredients determined in such a way have been added to the input list (i.e., ingredients) that the prediction algorithm has the task of processing and, therefore, both the original informative contributions and those amplified in the fictitious ingredient are subject to analysis.

### Management of Physical Conditions in Relation to the Static Properties

The quality of the predictions also depends upon a series of physical conditions that should be satisfied by the algorithm during the “training step”.

In particular, the predictions become more reliable when the model is forced to simultaneously satisfy predetermined physical constraints. In fact, materials science, corroborated by clear experimental evidence, teaches that for the same formulation, the estimate of the Stress-Strain curve varies as the experimental test conditions vary (see FIG. 5). These variations may assume a very complicated nature and therefore the identification of methods that are able to estimate and apply them implicitly and automatically may be great help. Data Driven modelling applications, by virtue of Machine Learning algorithms, have effectively given the possibility to estimate and impose the necessary physical constraints that describe the different experimental test conditions and their mutual relationships. Such constraints are enforced through the application of the machine learning algorithm stack.

In particular, with reference to FIG. 2, which shows a schematization of the stack of algorithms used, the second layer of the model introduces and applies the physical constraints.

More specifically, the first layer of the Machine Learning algorithm stack has been developed to provide a first estimate of the static properties to be predicted. In fact, in this layer of the stack, dedicated Machine Learning algorithms will be developed and trained in order to make predictions of the static properties for each of the physical test conditions considered. For this purpose, the algorithms (i.e. modules Mᵢ, Mⱼ, Mₖ, Mₗ in FIG. 2) are trained with the following inputs:
1. recipes: all the recipes/formulations (i.e. all the quantities of the ingredients) are supplied as input;
2. physical properties.

Differently, the second layer of the Machine Learning algorithm stack has been developed to be able to impose the physical constraints and therefore to provide an optimal estimation of the static properties since a physical consistency has been “taught” to the model itself, being fed with the following inputs:
1. recipes: all the recipes/formulations (i.e. all the quantities of the ingredients) are supplied as input
2. physical constraints: the static properties corresponding to all the studied physical test conditions are provided as input. In the step of using the tool and therefore of real prediction of the properties, these inputs will correspond to the properties predicted in the previous layer of the stack.

In conclusion, since the Machine Learning algorithms belonging to the second layer of the stack have been trained to predict specific outputs, having as input the totality of the outputs corresponding to the different physical test conditions, they are able to automatically infer what are the differences between outputs that depend on the physical test conditions, i.e. are capable of giving rise to an automatic and implicit learning of the necessary physical constraints.

The second layer is designed to perform the final estimation of the physical properties. For this purpose, it is trained using:
first estimation of physical properties
physical properties
to perform the prediction of Physical Properties (i.e. their final estimates).

Therefore, the second layer of the Machine Learning algorithm stack performs a refinement of the predictions made by the algorithms in the first layer of the stack, by virtue of the implicit imposition of physical constraints related to the different experimental test conditions.

The purpose of this procedure is to promote models that are capable of making predictions that respect the physical constraints.

The present invention has heretofore been described with reference to the preferred embodiments thereof. It is intended that each of the technical characteristics implemented in the preferred embodiments described herein, purely by way of example, may advantageously be combined, in ways other than that described heretofore, also with other characteristics in order to give form to other embodiments which also belong to the same inventive nucleus and that all fall within the scope of protection afforded by the claims recited hereinafter.

1-7. (canceled)

8. A computer-implemented method for prediction of static properties of a composite to be tested for production of tire tread compounds, the method comprising:
providing a raw data database, wherein data in the raw data database comprises recipes for already existing composites and of corresponding known dynamic properties, to be used as a reference;
normalizing the data contained in the raw data database according to an iterative procedure;
pre-processing the normalized data by data mining to eliminate aberrant data and to add new fictitious ingredients relating to specific categories of actual ingredients;
training an algorithm based upon automatic learning using the pre-processed data, wherein the algorithm comprises at least two modelling layers operating in sequence, a first layer aimed at taking into account different temperature and aging conditions of the composite to be tested, and a second layer which introduces and applies physical constraints; and
applying the trained algorithm to a set of experimental data that are representative of the recipe of the composite to be tested, for prediction of the static properties of the composite to be tested.

9. The method of claim 8, wherein the static properties comprise a modulus at different strain levels, an elongation at break, and the modulus at break deriving from a stress-strain curve obtained by applying different test conditions.

10. The method of claim 8, wherein the raw data database contains data representative of a plurality of experimental measurement sessions.

11. The method of claim 10, wherein the normalization step comprises an iterative normalization based, at each iteration, upon a recipe that is most repeated in the raw data database, to perform connections between the experimental sessions in order to reduce a variability thereof, and reducing them to the same reference.

12. The method of claim 10, wherein the iterative normalization step is performed by dividing each of the static properties to be predicted by a corresponding property of the iteratively selected composites used as a reference, wherein the reference recipe constitutes a connection between various experimental sessions and enables comparison of these sessions.

13. The method of claim 8, wherein the pre-processing step comprises the application of data mining algorithms.

14. The method of claim 12, wherein the data mining algorithms perform removal of anomalous data and/or execution of Principal Component Analysis (PCA) to add new fictitious ingredients relating to specific categories of actual ingredients.
